(19) United States
(12) Patent Application Publication
(10) Pub. No.: US 2008/0234553 A1
(43) Pub. Date: Sep. 25, 2008

(54) NON-INVASIVE HUMAN-HEALTH-MEASUREMENT SYSTEM AND METHOD

(76) Inventors: David A. Urman, Portland, OR (US); Wesley Spiegel, Richardson, TX (US)

Correspondence Address: KOLISCH HARTWELL, P.C., 520 SW YAMHILL STREET, Suite 200, PORTLAND, OR, 97204, US

(21) Appl. No.: 12/052,486

(22) Filed: Mar. 20, 2008

Related U.S. Application Data
(60) Provisional application No. 60/895,947, filed on Mar. 20, 2007.

(51) Int. Cl. A61B 5/00

(52) U.S. Cl. 600/300

(57) ABSTRACT

Methods and hand-held devices non-invasively measure a health parameter in an individual or a plurality of individuals. Plural hand-held devices may be provided to plural individuals. Each device may compute a score based on the assayed health parameter in the individual. A server on a computer network may receive the scores and determine a pattern based on the scores. The hand-held devices may be further configured to receive additional data from the individuals regarding activities that may affect the individuals' scores. The server may receive the additional data from the devices and determine additional patterns.

### CROSS REFERENCE TO RELATED APPLICATIONS

This application claims the benefit of and priority under 35 U.S.C. § 119(e) to U.S. Provisional Patent Application No. 60/895,947 entitled “Non-Invasive Human-Health-Measurement System and Method,” filed Mar. 20, 2007, the disclosure of which is incorporated herein by reference.

### BACKGROUND

Many conventional health parameter tests are invasive and costly, both in terms of equipment and time. Further, obtaining a sample may be overly-invasive, as blood, plasma, urine, or other bodily fluids often are required.

Free radicals are atoms having at least one unpaired electron, making them highly reactive. Often referred to as “scavengers,” free radicals steal electrons from other atoms, thereby causing those other atoms to become free radicals. The newly formed free radicals in turn seek to steal electrons from yet other atoms, thereby resulting in a chain reaction.

Under normal conditions the antioxidant defense system within the body can easily handle free radicals that are produced. However, exercise, fatigue, or other factors may cause a rise in free radicals. Excessive amounts of free radicals in the human body have been implicated as playing a role in the etiology of cardiovascular disease, cancer, Alzheimer's disease, and Parkinson's disease. Some contend that undesirably-high free-radical levels are responsible for the aging process.

Due to the short half-lives of free radicals, direct assay of free-radical levels in a living body is difficult. Nevertheless, multiple methods of free-radical assay exist. One method utilizes electron spin resonance (“ESR”) indirectly to assay free-radical levels. However, this method, as with other conventional methods, suffers from inaccuracies due to poor sensitivity. Other approaches utilize markers related to free-radical levels. For instance, lipids subjected to free radicals produce lipid peroxide, which may be assayed. Other fatty acids may be peroxidized and broken down into aldehydes, also markers for the presence of free radicals. In general, these conventional tests have all been criticized for inaccuracies due to lack of specificity, sensitivity and reproducibility.

Examples of methods of assaying free-radical levels are described in U.S. Pat. Nos. 7,070,937 and 6,475,743, issued to Bar-Or et al., and U.S. Patent Applications 2005/0087452, to McAnalley et al., 2005/0202521, to Crum, 2006/0194333, to Pruche et al., 2005/244983, to Ching, 2005/142124, to Kaiser, and 2005/233471, to McEwan et al., the disclosures of which are incorporated herein by reference.

### BRIEF DESCRIPTION OF THE DRAWINGS

FIG. 1 shows one possible hand-held device used for assaying a health parameter in a living body.

FIG. 2 shows a possible flexible housing for use with the device shown in FIG. 1.

FIG. 3 depicts an example method.

FIG. 4 depicts a compound before and after exposure to free radicals.

### SUMMARY

A method is provided for monitoring a health parameter of plural individuals. The method includes the step of providing each individual with a non-invasive hand-held device configured to assay the individual for a marker related to the health parameter and compute a score based on the result of the assay, the score being indicative of the health parameter in that individual. The method also includes the steps of: receiving the score from each non-invasive hand-held device and storing the scores in a database; and determining a first pattern in the health parameter of the plurality of individuals based on the received scores.

Additionally, a hand-held device for non-invasively assaying a health parameter of an individual is provided. The device includes a cylindrical flexible housing having a first end, a second end, and a nominal diameter. The flexible housing is also configured to be pressed to a second diameter. The device also includes a frangible ampoule disposed within the flexible housing and configured to be broken when the flexible housing is pressed from its nominal diameter to the second diameter. A reagent is sealably disposed in the frangible ampoule and configured to react with a sample provided by the individual through the flexible housing to produce an indication of the presence of a marker of the health parameter. A filter is disposed adjacent the first end of the flexible housing, and it is configured to prevent the reagent from escaping the flexible housing while allowing passage of the sample provided by the individual into the flexible housing. The filter is positioned to maintain the frangible ampoule in a fixed position prior to its being broken.

The hand-held device also includes a control component that is configured to receive the second end of the flexible housing, and it includes a processor, memory, a display, and input controls. The control component also includes a detector that is constructed to detect the presence of the marker in a sample. The sample is made available for detection when the individual releases the reagent by pressing the flexible housing to break the ampoule, which provides a sample via the first end of the flexible housing. The processor is configured to compute a score based on detection of the marker. The detector may also take the form of means for detecting the presence of the marker.

Additionally, a hand-held device for non-invasively assaying a health parameter of an individual is provided. The device includes: a processor; memory; a display; and input controls. The device is also configured to: (i) receive a sample from a living body; (ii) detect in the sample the presence of a marker for the health parameter; (iii) compute a score based upon the detected marker; (iv) communicate the score to a server on a network; (v) receive additional data input by the individual using the input controls, the additional data indicating an activity on the part of the individual which affects the score; and (vi) communicate the additional data to the computer server.

### DETAILED DESCRIPTION OF VARIOUS EMBODIMENTS

Methods and hand-held devices are provided for non-invasively measuring a health parameter in an individual or a plurality of individuals. A plurality of hand-held devices may be provided to a plurality of individuals. Each device may compute a score based on the assayed health parameter in the individual. A server on a computer network may receive the scores and determine a pattern based on the scores. The hand-held devices may be further configured to receive additional data from the individuals regarding activities that may affect the individuals' scores. The server may receive the additional data from the devices and determine additional patterns. While a number of health parameters may be assayed, in exemplary embodiments, free radical levels are measured.

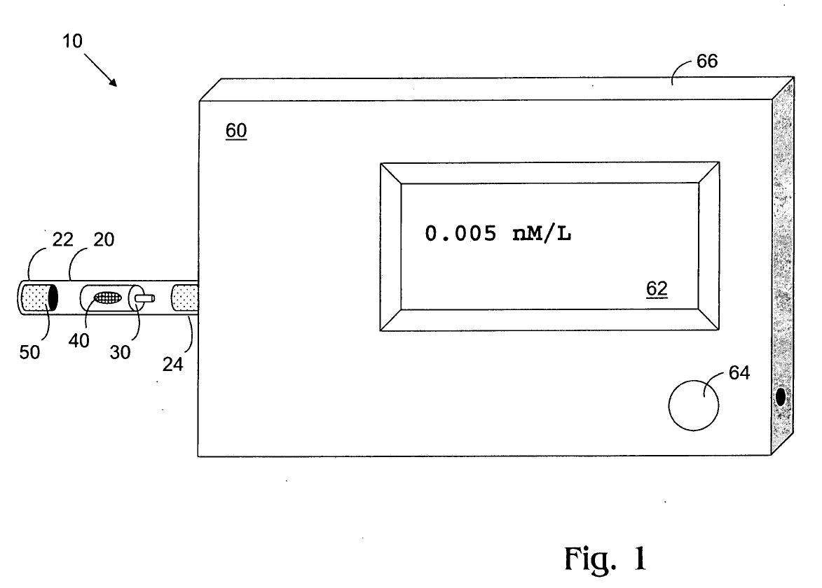

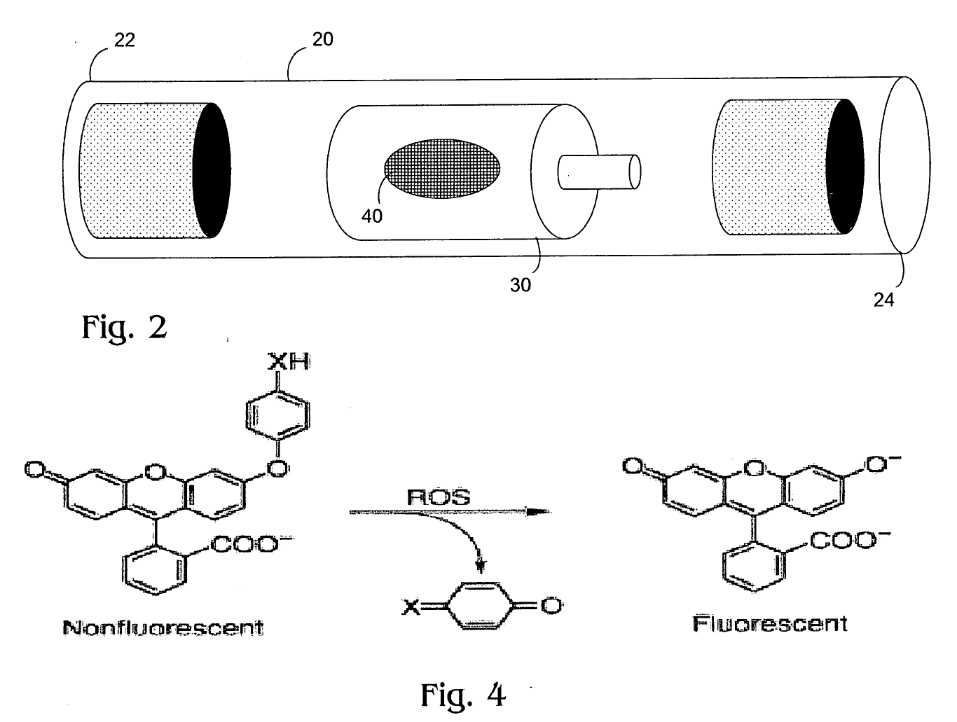

Referring now to FIG. 1, a hand-held device 10 for non-invasively assaying a health parameter of an individual includes a flexible housing 20, a frangible ampoule 30 disposed within the flexible housing and containing a reagent 40, a filter 50, and a control component 60.

Flexible housing 20 is shown having a cylindrical shape with a nominal diameter, but other shapes are possible. Flexible housing 20 may be made of a material which is flexible enough for a person to be able to press or squeeze it from its nominal diameter to a second diameter smaller than the nominal diameter. An individual may provide a biological sample (e.g., breath, saliva, blood) to flexible housing 20, as will be discussed further below.

Reagent 40 may be sealably disposed in frangible ampoule 30. Frangible ampoule 30 may be configured to be broken when flexible housing 20 is pressed from its nominal diameter to the second diameter.

Reagent 30 may be a chemical or compound configured to react with a sample provided by an individual through flexible housing 20 to produce an indication of the presence of a marker of a health parameter in the individual.

Flexible housing 20 has a first end 22 and a second end 24. A filter 50 may be disposed within flexible housing 20 adjacent to first end 22. Filter 50 may be configured to prevent reagent 40 from escaping the flexible housing 20 while allowing passage of the sample provided by the individual into flexible housing 20. Filter 50 further may be positioned to maintain the frangible ampoule 30 in a fixed position prior to its being broken.

Control component 60 may include a processor, memory, a display 62, input controls 64, power supply, a housing 66, and a detector constructed to detect the indication of the presence of the marker when the individual releases reagent 40, and provides a sample via the first end 22 of flexible housing 20. The detector may also take the form of means for detecting the presence of the marker.

Display 62 may be digital or a liquid crystal display (“LCD”), and it may be coupled to the power supply and the processor. In some embodiments, display 62 may be capable of displaying color. In other examples, particularly where display 62 is digital, display 62 may be monochromatic. In some embodiments, the processor performs instructions in memory that cause display 62 to prompt commands for input by the individual. These commands may tell the individual to input a standard of measurement, to insert a blank, to press one or more buttons, to insert a flexible housing containing a reagent, to enter additional information, or to press a “Get Results” button. Display 62 may also output information to the individual, including the results of the test such as the individual's score, or error messages.

The power supply may comprise a single 9-volt battery, or it may comprise two 3-volt batteries. The power supply may also come in the form of a rechargeable battery. Additionally and alternatively, the power supply may be a source of electricity available via a power cord plugged into a standard electrical outlet. In an exemplary embodiment, the power requirement is 9 volts.

For the purposes of being handheld, device 10 preferably will weigh less than 500 grams, including a battery. The preferred embodiment will weigh approximately 200 grams.

In some examples, reagent 40 may be configured to exhibit various colors when exposed to a component of the sample (e.g., change colors in the presence of free radicals or markers of free radical activity in exhaled breath condensate). In such cases, the means for detecting may include a calorimeter or other device for measuring color and communicating the measurement to the processor for storage in memory, computation of a score, and for output on display 62. In other examples, reagent 40 may exhibit characteristics other than color when exposed to a particular component of a sample, such as temperature, PH level, state, or the like. In such examples, the means for detecting may be an appropriate device for measuring such characteristics.

Control component 60 also may be configured to receive second end 24 of flexible housing 20. Once second end 24 is affixed to control component 60, a user may break ampoule 30 and release reagent 40 by squeezing flexible housing 20 from its nominal diameter to the second diameter. Then, the user may input a biological sample into flexible housing 20, for example by blowing into first end 22 of flexible housing 20. Reagent 40 may then react with a component of the sample (e.g., free radicals or a marker thereof, such as various aldehydes, contained in exhaled breath condensate) to exhibit a characteristic. In some embodiments, as discussed above, the characteristic is a particular color. In a preferred embodiment, the detection takes between two to four seconds to read the characteristic in the sample.

In some embodiments, a plurality of disposable flexible housings 20 may be provided with device 10 in order to provide the individual with multiple opportunities to use device 10, or to provide multiple individuals a safe, hygienic way to share a single device 10.

Once the processor of control component 60 has the result, it may compute a score based on the result. The computed score may be shown to an individual to notify the individual of the state of his or her health parameter.

In some embodiments, the processor is further configured to communicate the individual's score to a server on a computer network. Some devices 10 may be configured to connect to the computer network directly using wireless or wired methods. Other devices 10 may be configured to connect to a personal computer which itself is connected to the network. In such cases, the devices 10 may connect to the personal computer using various connection types, such as USB, FireWire or Bluetooth. The server may collect scores from multiple devices 10 as described in more detail below to compute statistical information about the health parameter in a plurality of individuals. For example, a server may take a plurality of scores and determine a pattern, such as a Gaussian curve. Such information may be useful to various parties, such as doctors, nutritionists, researchers or insurance companies.

In addition to the sample, from which control component 60 obtains an indication of the health parameter as described above, individuals may provide additional information to control component 60 via input controls 64. This additional information may include activity of which the individual partakes which may affect the health parameter in the individual, and hence, the individual's score. Such additional information may include but is not limited to information about physical activity partaken by the individual, dietary information about food consumed by the individual, information about alcohol consumed by the individual, information about tobacco use by the individual, and the like. For example, it has been documented that smokers typically have higher levels of free radicals. So if a smoker uses device 10 to measure his or her free radical levels, the smoker may indicate that he or she smokes, and the device may adjust the score and/or make recommendations to the smoker, such as to quit smoking.

In some embodiments, the processor of the control component 60 may communicate, in addition to the score, the additional information to the server on the computer network. The server may then use the score and additional information to perform further statistical analysis. For example, a server may use a plurality of scores and additional data to determine multiple patterns. Such patterns may include classifications. For example, individuals having high free radical levels who partake in regular exercise may be classified as athletes. Servers may be configured to communicate an individual's classification back to the individual via means such as email or through the individual's device 10.

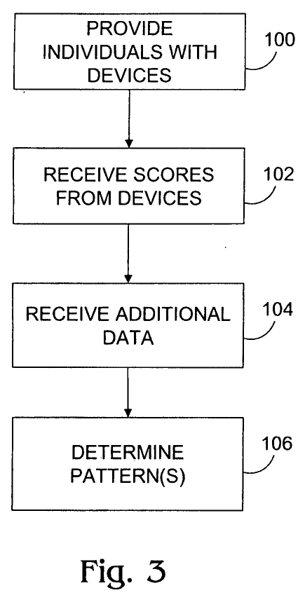

FIG. 3 depicts an example method for non-invasively assaying a health parameter in a plurality of individuals using hand-held devices such as those described above. In step 100, each individual is provided with a non-invasive hand-held device 10. In step 102, a score is received from each device 10 and stored in a database. In some embodiments, additional data such as that described above is also received in step 104. In step 106, patterns are determined based on the received scores and, in some embodiments, the received additional data.

While a number of health parameters may be assayed using the above-described devices and methods, a health parameter that is measured in many embodiments is the free radical level in each individual. The devices and methods may measure free-radical levels using saliva, breath, or any other sample that may be obtained non-invasively. When using breath as a sample, results may be obtained particularly quickly.

In some embodiments, fluorescence is used to perform the assay. Fluorescence is an optical phenomenon occurring when certain molecules—also known as fluorophores—absorb photons (i.e. are exposed to various forms of light). After absorbing photons, fluorophores emit other photons often having longer wavelengths than the absorbed photons. While the wavelengths of the absorbed photons may be within ranges invisible to the naked eye, the longer wavelengths exhibited by the emitted photons are often within the visible range. Fluorescent compounds will therefore emit visible light when exposed to various forms of light.

Some embodiments may assay free-radical levels in a living body using various compounds which become fluorescent only in the presence of free radicals. The amount of fluorescent light (i.e. photons with visible wavelengths) emitted from the compound is proportional to the amount of free radicals to which the compound is exposed. Thus, by exposing the compounds to bodily fluids (such as saliva or breath) which contain free radicals, the compounds become visibly fluorescent in proportion to the amount of free radicals in the bodily fluid.

FIG. 4 shows the three stages a molecule of one of the compounds described below might experience upon exposure to a free radical. On the left, the nonfluorescent molecule has at its upper right region an electron. The middle figure depicts a reactive oxygen species (“ROS”) coming into contact with the molecule and stealing the electron. The figure on the right shows the now fluorescent molecule without the electron.

Compounds that may be used in the described method include but are not limited to Aminophenyl Fluorescein (“APF”), Diphenyl-1-Pyrenylphosphine (“DPPP”), Nitro blue tetrazolium chloride (“NBT”), and 2,3-Diaminonaphthalene (“DAN”).

APF may be used as a ROS indicator. APF is nonfluorescent until it reacts with a hydroxyl radical or peroxynitrite anion. APF will also react with the hypochlorite anion (-OCI). In the presence of a specific ROS, APF yields a bright green-fluorescent product (excitation/emission maxima ˜490/515 nm) that is compatible with all fluorescence instrumentation capable of visualizing fluorescein.

DPPP is essentially nonfluorescent until oxidized to a phosphine oxide by peroxides. In vitro, DPPP remains nonfluorescent in the presence of hydroxyl radicals generated by the Cu2+-ascorbated method.

Tetrazolium salts, including NBT, are widely used for detecting the redox potential of cells for viability, proliferation and cytotoxicity assays. Upon reduction, these water-soluble colorless compounds form uncharged, brightly colored formazans. Several of the formazans precipitate out of solution and are useful for histochemical localization of the site of reduction or, after solubilization in organic solvent, for quantitation by standard spectrophotometric techniques.

In a reaction similar to that of DAF-FM, 2,3-diaminonaphthalene reacts with the nitrosonium cation that forms spontaneously from nitric oxide to form the fluorescent product, 1H-naphthotriazole.

In addition to the above compounds, any compound capable of fluorescing in the presence of free radicals may be used.

Additionally, while saliva and breath are examples of biological samples used to practice the method, any biological samples, including blood, plasma, sweat, tears, mucus, urine, bowel movement, semen, or any other substance produced by a living body, also may be used. In the preferred embodiment, the sample used is obtained using non-invasive techniques, such as by collecting breath condensate in flexible housing 20.

Accordingly, while embodiments of election methods and systems have been particularly shown and described with reference to the foregoing disclosure, many variations may be made therein. Various combinations and sub-combinations of features, functions, elements and/or properties may be used. Such variations, whether they are directed to different combinations or directed to the same combinations, whether different, broader, narrower or equal in scope, are also regarded as included within the subject matter of the present disclosure. The foregoing embodiments are illustrative, and no single feature or element is essential to all possible combinations that may be claimed in this or later applications. The claims, accordingly, define selected inventions disclosed in the foregoing disclosure. Where the claims recite “a” or “a first” element or the equivalent thereof, such claims include one or more such elements, neither requiring nor excluding two or more such elements. Further, ordinal indicators, such as first, second or third, for identified elements are used to distinguish between the elements, and do not indicate a required or limited number of such elements, and do not indicate a particular position or order of such elements unless otherwise specifically stated.

What is claimed is:

1. A method of monitoring a health parameter of a plurality of individuals, the method comprising the steps of:
providing each individual with a non-invasive hand-held device configured to assay the individual for a marker related to the health parameter and compute a score based on the result of the assay, the score being indicative of the health parameter in that individual;
receiving the score from each non-invasive hand-held device and storing the scores in a database; and
determining a first pattern in the health parameter of the plurality of individuals based on the received scores.

2. The method of claim 1, further comprising the steps of:
determining a second pattern in the health parameter of the plurality of individuals; and
categorizing each individual as falling within the first or second pattern based on the individual's score.

3. The method of claim 1, further comprising the step of receiving additional data from each non-invasive hand-held device indicating activity on the part of the individual using the non-invasive hand-held device which affects the individual's score, wherein the first pattern is further based on the additional data.

4. The method of claim 3, wherein the additional data received from each non-invasive hand-held device includes information about physical activity partaken by the individual using that device.

5. The method of claim 3, wherein the additional data received from each non-invasive hand-held device includes information about vitamin supplements consumed by the individual using that device.

6. The method of claim 3, wherein the additional data received from each non-invasive hand-held device includes dietary information about food consumed by the individual using that device.

7. The method of claim 3, wherein the additional data received from each non-invasive hand-held device includes information about alcohol consumed by the individual using that device.

8. The method of claim 3, wherein the additional data received from each non-invasive hand-held device includes information about tobacco use by the individual using that device.

9. The method of claim 1, wherein the health parameter includes free radical levels of each individual.

10. A hand-held device for non-invasively assaying a health parameter of an individual, the device comprising:
a cylindrical flexible housing having a first end, a second end, and a nominal diameter, the flexible housing being configured to be pressed to a second diameter;
a frangible ampoule disposed within the flexible housing and configured to be broken when the flexible housing is pressed from its nominal diameter to the second diameter;
a reagent sealably disposed in the frangible ampoule and configured to react with a sample provided by the individual through the flexible housing to produce an indication of the presence of a marker of the health parameter;
a filter disposed adjacent the first end of the flexible housing, the filter being configured to prevent the reagent from escaping the flexible housing while allowing passage of the sample provided by the individual into the flexible housing, the filter being positioned to maintain the frangible ampoule in a fixed position prior to its being broken; and
a control component, configured to receive the second end of the flexible housing, the control component having a processor, memory, a display, input controls, and a detector constructed to detect the presence of the marker when the individual releases the reagent by pressing the flexible housing to break the ampoule, and provides a sample via the first end of the flexible housing, and wherein the processor is configured to compute a score based upon detection of the marker.

11. The hand-held device of claim 10 further configured to receive additional data via the input controls from the individual including activity that affects the individual's score.

12. The hand-held device of claim 11 wherein the additional data received from the individual includes information about physical activity partaken by the individual.

13. The hand-held device of claim 11 wherein the additional data received from the individual includes dietary information about food consumed by the individual.

14. The hand-held device of claim 11 wherein the additional data received from the individual includes information about alcohol consumed by the individual.

15. The hand-held device of claim 11 wherein the additional data received from the individual includes information about tobacco use by the individual.

16. The hand-held device of claim 10, wherein the processor is further configured to communicate the individual's score to a server on a computer network.

17. The hand-held device of claim 16, wherein the processor is further configured to:
receive additional data from the individual including activity that affects the individual's score;
communicate the additional data to the server on the computer network; and
receive, in response to the communicated score and additional data, a classification of the individual.

18. A hand-held device for non-invasively assaying a health parameter of an individual, the device comprising:
a processor;
memory;
a display; and
input controls;
wherein the device is configured to:
receive a sample from a living body;
detect in the sample the presence of a marker of the health parameter;
compute a score based on the detected marker;
communicate the score to a server on a network;
receive additional data input by the individual using the input controls, the additional data indicating an activity on the part of the individual which affects the score; and
communicate the additional data to the computer server.

19. The hand-held device of claim 18 further configured to receive from the computer server, a classification based on the individual's score and the additional data.

20. The hand-held device of claim 18 wherein the data received from the individual includes at least information about physical activity conducted by the individual, dietary information about food consumed by the individual, information about alcohol consumed by the individual or information about tobacco used by the individual.
